(19) United States
(12) Patent Application Publication
(10) Pub. No.: US 2002/0138072 A1
(43) Pub. Date: Sep. 26, 2002

(54) Handpiece for projecting laser radiation in spots of different color and size

(76) Inventors: John F. Black, San Mateo, CA (US); David G. Angeley, San Jose, CA (US); R. Russell Austin, Half Moon Bay, CA (US)

Correspondence Address: EITAN, PEARL, LATZER & COHEN-ZEDEK, ONE CRYSTAL PARK, SUITE 210, ARLINGTON, VA, 22202-3709, US

(21) Appl. No.: 09/815,467

(22) Filed: Mar. 23, 2001

(51) Int. Cl. A61B 18/20

(52) U.S. Cl. 606/10; 606/17

(57) ABSTRACT

An optical system is arranged to deliver electromagnetic radiation in first and second different wavelengths to tissue to be treated therewith. The optical system forms the different-wavelength radiations into beams of different sizes and combines the beams on a common optical path. The different-wavelength radiations on the common optical path are projected by the optical system onto the tissue to be treated to form overlapping treatment spots of different sizes. In one example, the treatment spots are concentric and the size of the treatment spots and the ratio of the treatment-spot sizes can be selectively varied.

### TECHNICAL FIELD OF THE INVENTION

The present invention relates in general to devices for delivering laser radiation in laser treatment of blood vessels. The invention relates in particular to a device which projects laser radiation of different wavelengths into overlapping treatment spots having a size dependent on the wavelength.

### DISCUSSION OF BACKGROUND ART

In a paper “Cooperative Phenomena in Two-Pulse, Two-Color Laser Photocoagulation of Cutaneous Blood Vessels”, Black et al. Proc SPIE 4244A-02, (2001), (in press), a system for treating defective blood vessels is disclosed. In this treatment, two pulses of different wavelengths are delivered in a predetermined time sequence to a blood-vessel. In certain embodiments of the treatment, the time sequence and length of the pulses is such that the pulses overlap in time. The pulses act in a synergistic manner to effect permanent blood vessel damage at radiant exposures where the two pulses individually would have little or no effect. Once the blood vessel is damaged it is eventually reabsorbed by the body and replaced with scar tissue.

The reduced radiant exposures offered by the treatment significantly reduce the chance of a patient feeling pain during the procedure. The reduced radiant exposures also significantly reduce the possibility of collateral damage to tissue outside the treatment area Preferred wavelengths for the two pulses are about 532 nanometers (nm) and 1064 nm. In color terms, one pulse can be characterized as being a green pulse and the other a near-infrared (NIR) pulse. Briefly, The green pulse is delivered first and preconditions the blood vessel to a point where blood therein can be completely coagulated by the NIR pulse. The green and NIR wavelengths have different propagation characteristics in skin tissue and in blood in vessels in the tissue. Green radiation is strongly scattered in tissue and is strongly absorbed in normal blood. Because of this, in the Black et al. treatment, it is preferable to cover as much of the periphery of a vessel as possible with the green radiation. In this way, radiation can reach the sides of the vessel through a scattering process. The NIR radiation is scattered significantly less in the tissue than the green radiation and is less strongly absorbed in the blood. NIR radiation delivered to surrounding tissue has no therapeutic effect and could cause patient discomfort. Accordingly it is believed that the Black et al. treatment would benefit from delivering green radiation over a larger area of tissue than the area of tissue to which NIR radiation is delivered.

In laser dermal and vascular treatments, laser radiation is preferably delivered from a laser supplying the radiation via an optical fiber to a handpiece. The handpiece includes an optical system for projecting the radiation into a well-defined spot, and, as the name suggests, can be conveniently held by a surgeon and used to direct the radiation spot to a treatment site.

There is a need for such a conveniently holdable handpiece for use in the Black et al. treatment. Preferably, the handpiece should include an optical system which is capable of receiving radiation of two different colors (wavelengths) and projecting the radiation into two overlapping, preferably concentric, spots having a different size for the different colors. Accordingly, the handpiece should preferably be capable of selectively varying the treatment-spot sizes within a range thereof, at least with a fixed ratio of one to the other. It would also be of advantage if the treatment-spot size-ratio were also variable. The optical system should preferably, also, be substantially telecentric. This would provide for minimizing changes in spot-characteristics as a function of the distance (working distance) of the handpiece from the vessel being treated.

### SUMMARY OF THE INVENTION

In one aspect of the present invention, the above discussed requirements of a two-wavelength projecting handpiece can be satisfied by incorporating in the handpiece an optical system comprising first and second optical subsystems. The different color radiations can be described as radiations having first and second different wavelengths. The first optical subsystem is arranged to form the different color radiations received from sources thereof into beams of first and second different sizes, and combining the different sized beams on a common optical path. The second optical subsystem is arranged to project the combined, different-sized beams onto the tissue to be treated to form overlapping treatment spots thereon of respectively third and fourth different sizes. Preferably, the first and second optical subsystems are arranged such that the treatment spots are about circular and are concentric.

In another aspect of the present invention. At least one of the optical subsystems is arranged such that the treatment-spot sizes are selectively variable. The optical subsystems can arranged such that the treatment-spot sizes can be varied in a fixed or variable ratio one to the other.

In yet another aspect of the invention, the first optical subsystem includes first i s and second optical fibers for delivering respectively the first and second-wavelength radiations to the optical system. The first and second optical fibers have respectively first and second exit-face sizes corresponding to the first and second beam sizes. The first and second optical fibers may be arranged to transport the first and second-wavelength radiations from the corresponding sources thereof into the first optical subsystem. In one alternative arrangement, the first and second-wavelength radiations may be delivered to the first optical subsystem from the corresponding sources thereof along the first optical fiber.

In another alternative arrangement, the first and second radiations may be delivered to the first optical subsystem along a third optical fiber. In this case, the first optical subsystem includes a wavelength selective splitter for extracting the first and second-wavelength radiations from the first optical fiber and directing the first and second wavelength radiations into respectively the first and second optical fibers.

### BRIEF DESCRIPTION OF THE DRAWINGS

The accompanying drawings, which are incorporated in and constitute a part of the specification, schematically illustrate a preferred embodiment of the present invention, and together with the general description given above and the detailed description of the preferred embodiment given below, serve to explain the principles of the invention.

FIGS. 1A, 1B, and 1C schematically illustrate different arrangements wherein a source of short wavelength optical radiation and a source of long wavelength optical radiation deliver the short and long wavelength radiations to a handpiece including an optical system in accordance with the present invention.

FIG. 2 is a perspective view schematically illustrating one preferred embodiment of an optical system in accordance with the present invention, including an optical subsystem having partially-separate optical paths for NIR radiation and 532 nm radiation, and arranged to receive both radiations from a common optical fiber, the system providing for variable treatment-spot sizes in a fixed size-ratio.

FIG. 3A is an elevation view schematically illustrating detail of the path of NIR radiation through the optical system of FIG. 1.

FIG. 3B is an elevation view schematically illustrating detail of the path of 532 nm radiation through the optical system of FIG. 1.

FIGS. 3C-E depict in tabular form a preferred prescription for optical components and spacings thereof in the optical system of FIG. 1.

FIG. 4 is a perspective view schematically illustrating another preferred embodiment of an optical system in accordance with the present invention, including an optical subsystem having partially-separate optical paths for NIR radiation and 532 nm radiation, and arranged to receive the NIR radiation and 532 nm radiations, separately, from respectively first and second optical fibers, the system providing for variable treatment-spot sizes in a fixed size-ratio.

FIG. 5A is an elevation view schematically illustrating detail of the path of NIR radiation through the optical system of FIG. 4.

FIG. 5B is an elevation view schematically illustrating detail of the path of 532 nm radiation through the optical system of FIG. 4.

FIGS. 5C-E depict in tabular form a preferred prescription for optical components and spacings thereof in the optical system of FIG. 4.

FIG. 6 schematically illustrates yet another preferred embodiment of an optical system in accordance with the present invention, similar to optical system of FIG. 4 but arranged for providing for selectively variable treatment-spot sizes in a selectively variable size-ratio.

FIG. 7 schematically illustrates yet another preferred embodiment of an optical system in accordance with the present invention, similar in principle to the optical system of FIG. 4 but configured to provide fixed treatment-spot sizes in a fixed size-ratio.

FIG. 8 schematically illustrates still another preferred embodiment of an optical system in accordance with the present invention, including an optical subsystem having separate optical paths therethrough for NIR radiation and 532 nm radiation, and arranged to receive the NIR radiation and 532 nm radiation, separately from respectively first and second optical fibers, the system providing for fixed treatment-spot sizes in a fixed size-ratio.

FIG. 9 schematically illustrates a further preferred embodiment of an optical system in accordance with the present invention, including an optical subsystem having a common optical path therethrough for NIR radiation and 532 nm radiation, and arranged to receive both radiations from a common optical fiber, the system providing for fixed treatment-spot sizes in a fixed size-ratio.

### DETAILED DESCRIPTION OF THE INVENTION

The present invention is discussed in the context of projecting, from a single handpiece, overlapping different sized spots of two different wavelengths of electromagnetic (optical) radiation. This terminology should be understood to include ultraviolet, visible and infrared radiation. The terminology “different-color radiation” may be used, for convenience as an alternative to “different wavelengths of electromagnetic radiation”. The two different colors or wavelengths may also be referred to as short wavelengths and long wavelengths. Specific examples of certain embodiments of the optical system are described with reference to 532 nm as the short wavelength radiation and 1064 nm as the long wavelength radiation. These are preferred wavelengths in the above described Black et al. treatment. These wavelengths should by no means be considered as limiting the present invention.

Similarly, while lasers are preferred sources of the electromagnetic radiation in the Black et al. and other prior art vascular treatments, the present invention should not be construed as being limited to use with laser radiation. Other electromagnetic radiation sources, the output of which can be transported by an optical fiber, for example a gas-discharge lamp, is applicable in the present invention. The Black et al. treatment is described in detail in co-pending application Ser. No. 09/538,787, assigned to the assignee of the present application, and the complete disclosure of which is hereby incorporated by reference.

Optical fibers are described hereinbelow as being used to transport the electromagnetic radiation. In certain embodiments of the inventive optical system, optical fibers of different sizes may be used to contribute to forming the radiation into beams of different sizes for radiation colors. Where the size of optical fibers is discussed herein what is meant is the core size of the optical fiber.

In embodiments of optical systems discussed below it is preferable that the optical systems be substantially telecentric on the image (treatment) side thereof. A substantial degree of telecentricity provides that the shape of treatment spots projected by the systems does not vary unacceptably with changes in working distance of the optical system from tissue being treated. A common measure of telecentricity, in terms of conventional ray tracing, is the slope of a principal ray with respect to the optical axis on the image side of the optical system. In a perfectly telecentric optical system, this slope would be zero, i.e., the principal ray would be parallel to the optical axis of the optical system. In a less-than-perfectly-telecentric system the (non-zero) slope of the principal rays can be used as a measure of the degree of telecentricity. Here, of course a lower number indicates greater telecentricty. In a paper “Optical Design and Specification of Telecentric Optical Systems”, Michael A. Pate, SPIE Vol 3482, pp 877-886 1998 a recommended definition of telecentricity is a principal ray slope of less than one degree (17 milliradians).

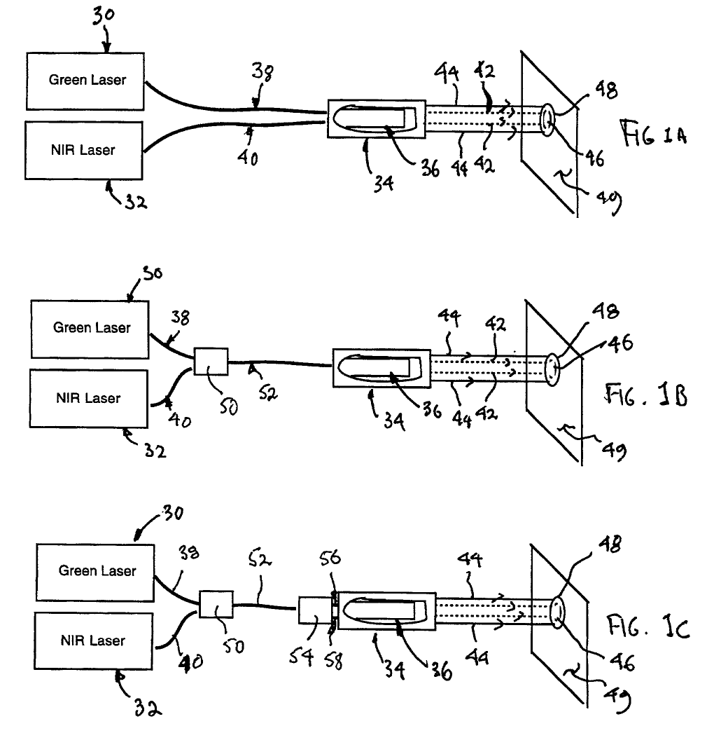

Turning now to the drawings, wherein like features are designated by like reference numerals, FIGS. 1A-C schematically illustrate different arrangements wherein a source 30 of short-wavelength optical radiation and a source 32 of long-wavelength optical radiation deliver the short and long wavelength radiations via one or more optical fibers to a handpiece 34 (shown partly cutaway). Handpiece 34 includes an optical system 36 in accordance with the present invention. Source 30 is designated as a green laser, for example a 532 nm laser. Source 32 is designated an NIR laser, for example a 1064 nm laser. Those skilled in the art will recognize that it is also possible to generate and deliver both of these wavelengths from, for example, a frequency doubled Nd:YAG laser. Those skilled in the art will also recognize without detailed description or depiction how such a laser may be adapted to modify one or more of the arrangements of FIGS. 1A-C.

In the arrangement of FIG. 1A, green and NIR laser-radiations from lasers 30 and 32 are transported to optical system 36 through optical fibers 38 and 40 respectively. In this arrangement, optical system 36 is contemplated as being arranged to receive and project radiation from the separate fibers, which, as discussed further hereinbelow, may be of the same or different sizes. Optical system 36 projects the different-color radiations into overlapping spots. Here NIR radiation 42 is formed into a treatment spot 46 on tissue 49 to be treated. Green radiation 44 is formed into a spot 48 on the tissue, overlapping and concentric with spot 46. Green treatment spot 48, here, is larger than NIR treatment spot 46, as preferred in the Black et al. treatment. Those skilled in the art will recognize, from descriptions of embodiments of the present invention presented below that the long-wavelength spot may be made larger that the short-wavelength spot without departing from the spirit and scope of the present invention.

It has been found that in the Black et al, treatment, the size of treatment-spot 48 is preferably bigger (for example about two to three times bigger) than the diameter of a blood vessel being treated. This allows radiation to reach the sides of the vessel through a scattering process in the surrounding tissue. Up to a size of about 6.0 millimeters, the larger the spot size the deeper the penetration of the radiation. Preferably, treatment-spot 48 has a diameter about 4 mm or greater. Treatment-spot 46 preferably has a diameter no greater than about twice the diameter of a blood vessel being treated but with a minimum size of about 2 mm being preferred. This is because scattering of NIR radiation in tissue, while lower than that of green radiation, is not insignificant. Suitable ratios of the size of green and NIR spots 48 and 46 in a two-color vascular treatment such as that described in Black et al. are believed to be in a range from as low as about 6:5 up to 2:1 or even greater.

In FIG. 1B, optical fibers 38 and 40 are directed to a beam combiner 50 which directs the green and NIR laser radiation along a common optical fiber 52 to handpiece 34. Optical system 36 in this case is arranged to receive the radiations from the single fiber and form the radiations into spots of different sizes as depicted.

In FIG. 1C, optical fibers 38 and 40 are again directed to a beam combiner 50 which directs the green and red laser radiation along a common optical fiber 52. Adjacent handpiece 34, a wavelength-selective decoupler 54 separates the green and NIR wavelengths and directs the green wavelength into a short length of optical fiber 56 and the NIR wavelength into a short length of optical fiber 58.

In one arrangement of decoupler 54, either optical fiber 56 or optical fiber 58 may be simply an extension of optical fiber 52. As optical arrangements for multiplexing and demultiplexing different wavelengths into and out of optical fibers are well known in the art, such arrangements are not described in detail herein.

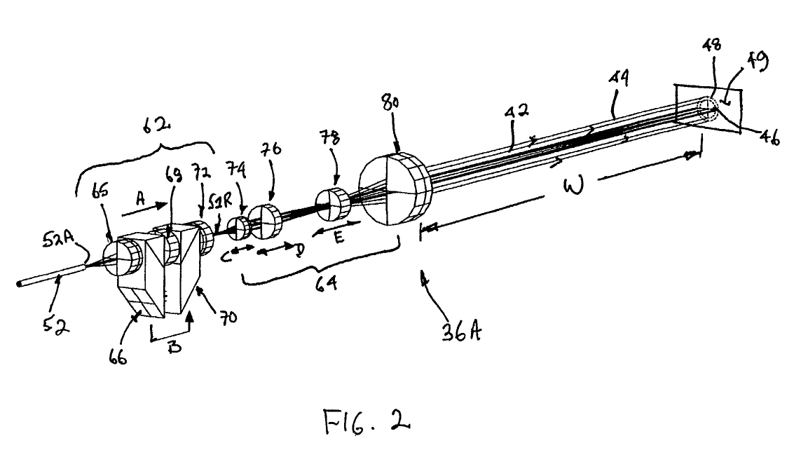

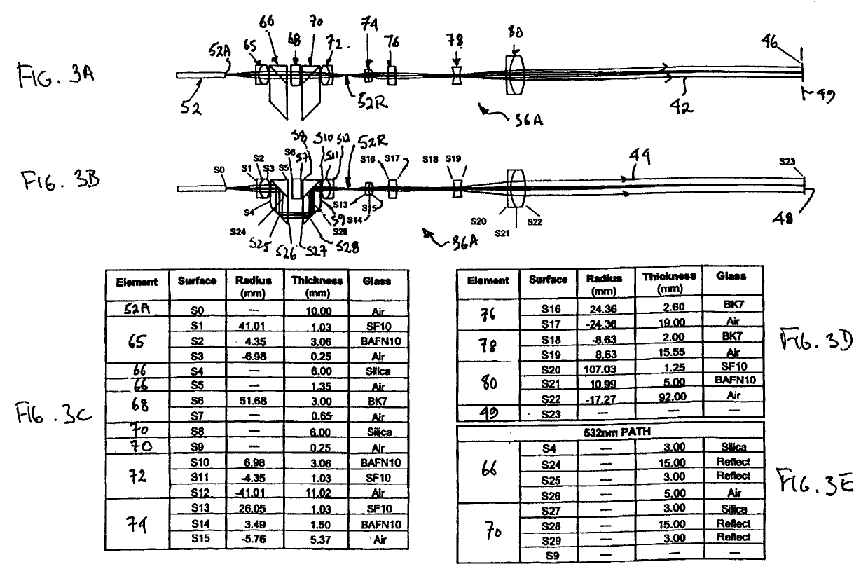

Referring now to FIG. 2, and additionally to FIGS. 3A and 3B, one preferred embodiment 36A of an optical system in accordance with the present invention is arranged to be cooperative with 532 nm and 1064 nm radiations received after being transmitted down a common optical fiber 52 as discussed above with reference to FIG. 1B. Optical system 36A includes an optical subsystem 62, here, in the form of a split-path offset-relay lens-group. Subsystem 62 is arranged such that 1064 nm (NIR) radiation passes therethrough in a direction indicated in FIG. 2 by arrow A. The 532 nm radiation passes through subsystem 62 in a direction indicated by arrow B. Optical system 36A also includes an optical subsystem 64, here, in the form of a substantially-telecentric, variable-magnification projection optical system.

Optical relay 62 includes a positive, cemented-doublet (two lens elements cemented together) lens 65, a beamsplitter rhomb 66, a positive singlet (one element) lens 68, another beamsplitter rhomb 70, and another cemented-doublet lens 72. Relay 62 relays two images (not explicitly depicted) of exit-face 52A into a position on the optical axis of optical system 36A generally indicated by reference numeral 52R.

Exit-face 52A of optical fiber is located at a distance greater than the focal length of lens 64 from lens 64. Light of each wavelengths emerges from exit-face 52A of optical fiber 52 as a diverging beam in which beams of both colors are coincident. Accordingly, beams of both wavelengths converge about equally as they leave lens 65. Surface S24 of rhomb 66 includes a multilayer dielectric coating arranged to reflect 532 nm radiation (substantially unpolarized) and transmit 1064 nm radiation (also substantially unpolarized). Accordingly, the paths of the two beams through the relay are separated into paths A and B.

The separated 532 nm and 1064 nm beams are recombined onto a common path at surface S29 of rhomb 70. Surface S29 includes a multilayer dielectric coating similar to the coating on surface S24 of rhomb 66. Path B of the 532 nm radiation, to far surface S29, does not encounter any optical components having optical power, either positive or negative. Path A, however, includes positive lens 68 and is optically shorter than path B. Accordingly, on recombination at surface 29 of rhomb 70, the 532 nm and 1064 nm beams have a different size and a different convergence. This is arranged, by suitable selection of component materials, surface curvatures, and spacings, such that at point 52R the intermediate image (not shown) of surface 52A in 532 nm (green) light is bigger than the intermediate image of the exit face on 1064 nm (NIR) light. As optical fiber 52, here, is assumed to have a circular cross-section the intermediate images are circular and concentric.

The intermediate images at point 52R become an object for zoom projection subsystem 64. Zoom subsystem 64 includes a positive doublet lens 74, a positive singlet lens 76, a negative singlet lens 78, and a positive doublet lens 80. Subsystem 64 is arranged to project magnified images 46, 48, (treatment-spots) of respectively the NIR and green intermediate images of point 52R in a treatment plane 49 at a working distance W from the optical system. The ratio of the image sizes, one to the other, will be essentially the same as the ratio of the sizes of the intermediate green and NIR images at point 52R.

The size of treatment-spots (images) 46 and 48 may be selectively varied by axially moving lenses 76 and 78 with respect to each other and with respect to lenses 72 and 80, as indicated in FIG. 2 by arrows D and E respectively. As the size of the treatment spots is varied, the ratio of treatment-spot sizes will stay essentially the same. Lenses 76 and 78 are depicted in FIG. 2 in about the relative positions thereof when the magnification of the spots is at about the middle of the magnification range of optical system 36A.

An exemplary specification for optical system 36A is depicted in tabular form in FIGS. 3C-E with reference to FIGS. 3A and 3B. FIGS. 3A and 3B depict respectively the path of NIR (44) and green (42) light rays through radiation 44 through the optical system. FIG. 3A indicates the lens elements by the reference numerals of FIG. 2. FIG. 3B indicates the lens elements by corresponding surface-designating numerals S1 through S8, and S10 through S22, for comparison with the specification tables of FIGS. 3C-E. In these tables, exit face 52A of optical fiber 52 is designated as surface SO, and treatment plane 49 is designated as surface S23.

The specification of FIGS. 3C-E indicates lens spacings selected to provide the sizes (diameters) of treatment-spots 46 and 48 of respectively about 3.34 and 4.55 mm. The diameter of optical fiber exit-face 52A is assumed to be 0.365 mm. This magnification of optical system 36A is about eleven times for the spacings indicated, which, in this example, is at about the middle of a range of magnifications selectively variable from about 5.5 times magnification to 22.0 times magnification. The system is substantially telecentric on the image side over most of this range, but falls off at the higher end of the range. From the specifications provided, one skilled in the optical design art using readily available commercial lens design software could determine the corresponding lens spacings required to provide smaller spotsizes.

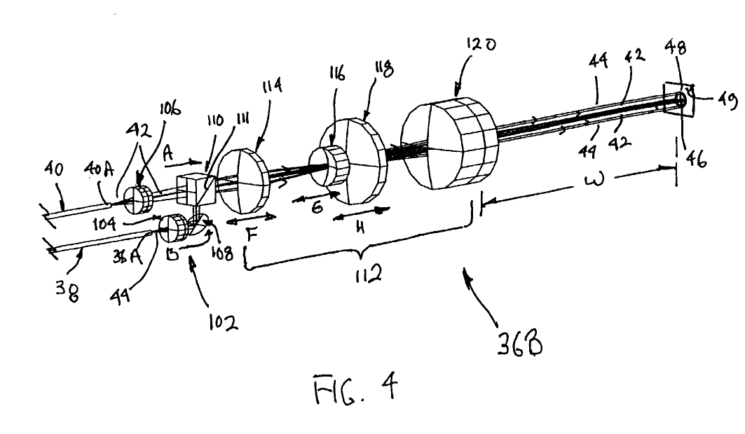

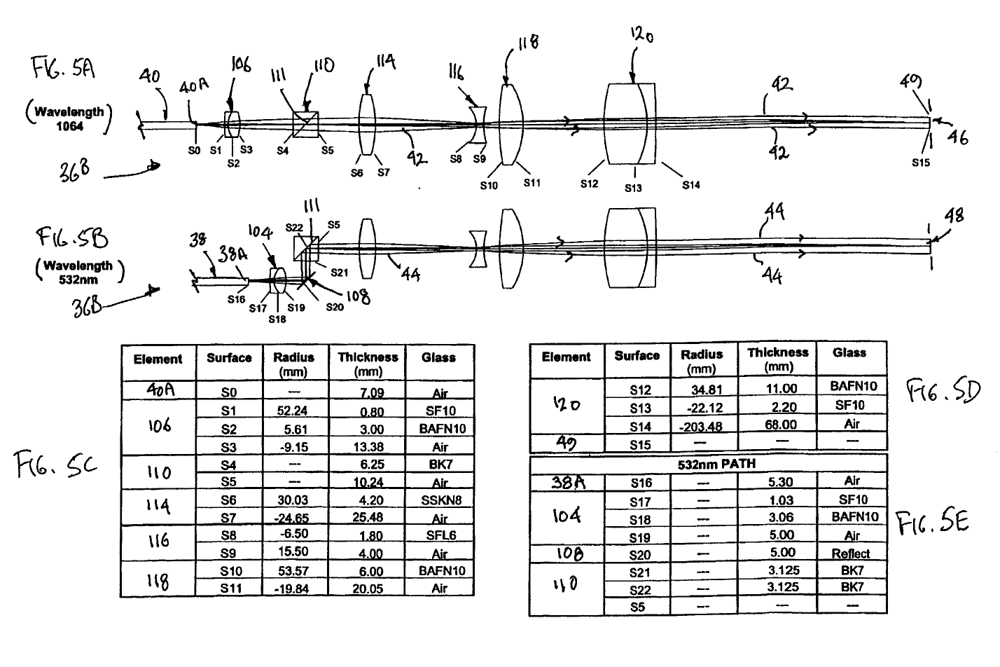

Referring now to FIG. 4, and additionally to FIGS. 5A and 5B, another preferred embodiment 36B of an optical system in accordance with the present invention is arranged to be cooperative with 532 nm radiation and 1064 nm radiation received after being transmitted through optical fibers 38 and 40 respectively (see FIG. 1A). Optical system 36B could also be cooperative with optical fibers 56 and 58 of FIG. 1C.

Optical system 36B includes an optical subsystem 102. Subsystem 102 includes positive doublet lenses 104 and 106, which, for purposes discussed further hereinbelow, preferably have different focal lengths. Optical system 36B also includes a front-surface mirror 108 and cube-beamsplitter (cemented biprism) 110 including a reflecting surface 111. Reflecting surface 111 includes a multilayer dielectric coating arranged to reflect 532 nm radiation and transmit 1064 nm radiation. It should be noted, here, at least for reasons discussed in detail further hereinbelow, that the optical fibers can be considered under certain circumstances as being part of optical subsystem 102.

A beam of 532 nm radiation 44 emerges from optical fiber exit-face 38A and enters optical system 36B via lens 104 of subsystem 102 as indicated by arrow B. A beam of 1064 nm radiation 42 emerges from optical fiber exit-face 40A and enters optical system 36B via lens 106 of subsystem 102, as indicated by arrow A. The 532 and 1064 nm radiations follow separate paths until they are combined into a common path by reflecting surface 111. The combined 532 nm and 1064 nm beams pass through an optical-subsystem 112.

Optical-subsystem 112 includes a positive singlet lens 114, a negative singlet lens 116, a positive singlet lens 118 and a positive doublet lens 120. Lenses 114, 116, and 118 are axially moveable with respect to each other and other optical components of the system as indicated in FIG. 4 by arrows F, G, and H respectively. This provides that the magnification of the optical system as a whole, and treatment-spot sizes projected thereby can be selectively variable.

It is preferable that the optical system as a whole be made substantially telecentric for both the 532 nm radiation path and the 1064 nm radiation path therethrough. Accordingly, the following general spacing relationships of optical elements are preferable.

Both lenses 104 and 106 should preferably be about confocal with subsystem 112, even if their focal lengths are different. Optical fiber end-face 38A should preferably be located at about a focal length of lens 104 from lens 104. Optical fiber end-face 40A should preferably be located at about (slightly less than) a focal of lens 106 from lens 106. The location of beamsplitter 110 and mirror 108 is not important from the point of view of optimizing telecentricity. However, it is preferable to locate the beamsplitter and mirror as close as possible to lenses 104 and 106, to provide a greater range of motion for lenses 114, 116 and 118, and, accordingly, a greater zoom (magnification) range for optical system 36B.

In optical system 36B as exemplified in FIGS. 4 and FIGS. 5A-E, optical fiber end-faces 38A and 40A are assumed to have the same diameter (about 0.365 mm). Lenses 104 and 106 are assumed to have different focal lengths, with the focal length of lens 104 being the shorter. The ratio of the focal lengths is that of the desired treatment-spot size-ratio. The focal length of lenses 104 and 106 being different, the sizes of the 532 nm and 1064 nm beams will be different as they are recombined by beamsplitter 110. At this point in the system, the 532 nm beam will actually be smaller than the 1064 nm beam. Optical subsystem 112 forms the different-sized beams into a green treatment spot 48 and a smaller NIR treatment spot 46. Treatment-spots 48 and 46 can be considered as being images of optical fiber end-faces 38A and 40A.

An exemplary specification for optical system 36B is depicted in tabular form in FIGS. 5C-E with reference to FIGS. 5A and 5B. FIGS. 5A and 5B depict respectively the path of NIR (42) and green (44) light rays through the optical system. In FIGS. 5A and FIG. 5B the lenses are indicated both by the reference numerals of FIG. 4 and by corresponding surface-designating numerals S1 through S14 and S17 through S22, for comparison with the specification tables of FIGS. 5C-E. In these tables, exit face 38A of optical fiber 38 is designated as surface S16, exit face 40A of optical fiber 40 is designated as surface SO, and treatment plane 49 is designated as surface S15.

The specification of FIGS. 5C-E indicates lens spacings selected to provide spot sizes (diameters) of treatment-spots 46 and 48 in the middle of the zoom range. In this example treatment-spots 46 and 48 have diameters of respectively about 2.77 mm and 3.70 mm. The diameter of optical fiber exit-faces 38A and 40A are assumed to be 0.365 mm. The magnification range of the example is from about 5.0 times to about 15.0 times.

From the description of optical system 36B provided above, one skilled in the art would recognize that the system could be modified in at least two relatively simple ways. In a first of these, the focal length of lenses 104 and 106 could be made the same and the diameter of optical fiber end-faces 38A and 40B, could be made different in the ratio of the desired difference in treatment-spot sizes. In the second of these, the focal length of lenses 104 and 106 could be made different and the diameter of optical fiber end-faces 38A and 40B, could also be made different. This could permit a greater treatment-spot size-ratio than might be practically possible by relying only on the focal lengths of lenses 104 and 106. In any variation of optical system 36B wherein the optical fibers diameters are different, the optical fibers themselves can be considered as being a functional part of optical subsystem 102.

In above-described embodiments 36A and 36B of the present invention, while the long and short wavelength treatment-spot sizes are selectively variable, the ratio of the treatment-spot sizes remains fixed as the treatment-spot sizes are varied. As noted above, it would be advantageous if both the different wavelength treatment-spot sizes and the ratio of those treatment-spot sizes could be selectively varied.

In FIG. 6, a modification 36C of optical system 36B is schematically illustrated. Optical system 36C includes an optical subsystem 142 corresponding functionally to optical subsystem 102 of optical system 36B of FIG. 4. Optical system 36C also includes an optical subsystem 144 corresponding functionally to optical subsystem 112 of optical system 36B. For simplicity of description, no ray traces are depicted. The paths of the different rays through optical system 36C are indicated simply by a common system axis 146 having NIR and green branches 146A and 146B thereof in optical subsystem 142. Corresponding lenses of optical systems 36C and 36B are designated by the same reference numerals to highlight the modification included in system 36C. Those skilled in the art will recognize that the corresponding lenses are not required to have the specifications of FIGS. 5C-E.

Selective variation of treatment-spot sizes in a fixed ratio is accomplished in optical system 36C by selectively, axially moving lenses 114, 116, and 118 in the same manner as in optical system 36B. In system 36C, lens 106 of optical system 36B is replaced with a two-lens group 150, comprising a positive lens 152 and a negative lens 154. Lenses 152 and 154 are selectively moveable with respect to each other as indicated by arrows J and K respectively. This in effect replaces the fixed lens 106 of optical system 36B with a lens group of variable focal length. Having this variable-focus group in only one of the radiation paths through optical system 36C provides that the ratio of treatment-spot sizes can be varied in addition to varying the sizes the treatment spots together.

A possible disadvantage of system 36C as depicted in FIG. 6 is that it can be difficult to maintain a desired degree of telecentricity in the path of the system that includes lens group 150 if the focal length of the group is varied substantially. This disadvantage could be remedied by providing a more complex construction of lens group 150. The advantage could also be remedied by providing that optical fiber 40 be also moveable cooperative with the movement of lenses 152 and 154. This could be accomplished for example, by means of an arrangement including a “floating” optical-fiber connector or the like incorporated in a handpiece 34.

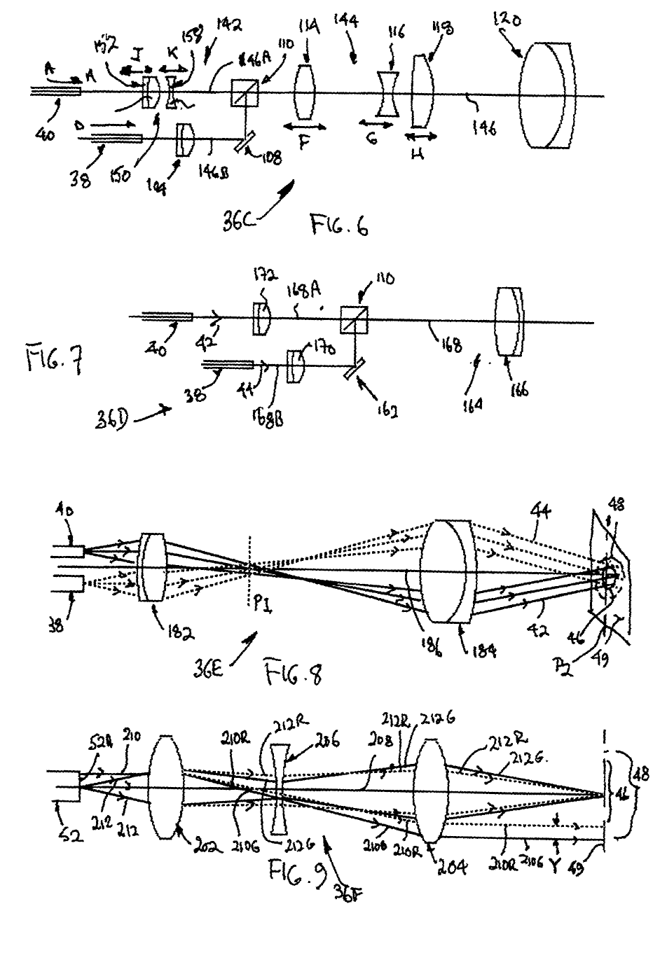

A capability to provide for selectively variable treatment-spot sizes is a particularly desirable feature of above described optical systems 36B and 36C. However, those skilled in the art, from the descriptions of these optical systems provided herein will recognize that the principle of the systems which provides for the treatment-spot sizes to be different is applicable in an optical system which provides treatment-spot sizes in a fixed size and in a fixed size-ratio. One example 36D of such an optical system is schematically depicted in FIG. 7.

Optical system 36D includes an optical subsystem 162 which is functionally equivalent to subsystem 102 of optical system 36B of FIG. 4. Optical system 36D also includes an optical subsystem 164, here, including only one doublet lens 166. Optical subsystem 144 corresponds functionally to optical subsystem 112 of optical system 36B except that the variable-magnification feature of optical system 36B is omitted. Again for simplicity of description, no ray traces are depicted. The paths of the different rays through optical system 36D are indicated simply by a common system axis 168 having NIR and green branches 168A and 168B thereof in optical subsystem 162.

Optical subsystem 162 includes lenses 170 and 172, mirror 108 and cube beamsplitter 110. Green and NIR radiations 44 and 42 enter optical system 36D via lenses 170 and 172 respectively. The paths of the green and NIR radiations are combined at cube beamsplifter 110. Green and NIR treatment-spot size differences can be provided by providing that lenses 170 and 172 have different focal lengths or providing that optical fibers 38 and 40 have different diameters, or both. Preferably, lenses 170 and 166 are spaced by about the sum of their focal lengths and lenses 172 and 166 are also spaced by about the sum of their focal lengths. This provides that the 532 nm and 1064 nm paths through the system are both substantially telecentric.

Referring now to FIG. 8, a further embodiment of an optical system 36E in accordance with the present invention includes doublet lenses 182 and 184 aligned on an optical axis 186. Optical fibers 38 and 40 delivering respectively green radiation 44 and NIR radiation 42 to the optical system are spaced apart transverse to axis 186 on opposite sides thereof. Optical fibers 38 and 40 have different numerical apertures and preferably also different diameters.

Optical fibers 38 and 40 are spaced apart from lens 182 by a distance greater than the focal length of the lens such that green and NIR radiation beams pass through the lens and are caused to cross each other at an intermediate pupil position P1 of optical system 36E. Tissue to be treated (treatment plane 49) is located at a conjugate (exit) pupil position P2 where beams 44 and 42 again cross and are overlapped to provide overlapping treatment spots 46 and 48 having a size-ratio about equal to the ratio of the corresponding optical fiber diameters.

The diameter of a beam at pupil P1 is about equal to the numerical aperture of the optical delivering the beam multiplied by twice the effective focal length of lens 182. The diameter of the beam at pupil P2 is equal to its diameter at P1 multiplied by the magnification provide by lens 184.

While optical system 36E is relatively simple and economical with respect to optical component count, compared with other embodiments of the present invention described above, it has some disadvantages. One such disadvantage is that, as the conjugate pupil does not correspond with a focal point of the system, treatment spots 46 and 48 may have somewhat less-than-sharp edges. A further such disadvantage is that the size and manner of overlap of the treatment spots may be sensitive to relatively small variations in spacing of then optical system from the treatment plane, i.e., variations in the working distance. Whether or not these disadvantages are acceptable will be determined, of course, by the intended use of the system.

FIG. 9 schematically depicts still a further embodiment 36F of an optical system in accordance with the present invention. Optical system 36F is configured to receive green and NIR radiation from the same optical fiber 52 (see FIG. 1B). Optical system 36F includes positive lenses 202 and 204 having a negative lens 206 therebetween. End-face 52A of the optical fiber and the lenses are aligned on a common optical axis 208. Optical system 36F configured to project images of exit-face 52A of optical fiber 50 with the image size (treatment spot 48) in green radiation being at least about 15% bigger than the image size (treatment spot 46) in NIR radiation. This can be accomplished by deliberately, grossly overcorrecting lateral color aberration of the system. The term “grossly overcorrecting”, here, means that the residual lateral-color aberration would be absolutely intolerable in a conventional multicolor or white-light imaging optical system. Lenses 202, 204, and 206 are fabricated from highly dispersive glasses. This is particularly important for lens 206. Surprisingly, it has been found that such a gross overcorrection can be accomplished while substantially correcting axial chromatic aberration sufficient that well defined treatment spots can be projected.

In FIG. 9, only sufficient rays are shown traversing optical system 36F to illustrate how the overcorrected lateral color and corrected axial color cause the different sized images to be formed. Green rays are depicted as solid lines and NIR rays as dashed lines. It should be noted that these lines depict only the estimated paths of rays for illustrating principles of optical system 36F.

The lateral color aberration is illustrated by tracing a paraxial ray 210 from the perimeter of optical exit-face 52A. After passing through lens 202, this ray angularly separates into a green ray 210G and an NIR ray 210R. The system is arranged such that this angular separation continues as far as lens 204. After traversing lens 204, rays 210G and 210R leave lens 204 parallel to axis 208 but laterally separated by a distance Y which corresponds to a difference in radius of the green and NIR treatment spots.

Axial color correction is illustrated by tracing marginal rays 212, through optical system 36F. After passing through lens 202 these rays also angularly separate into green (212G) and NIR (212G) rays. Optical system 36F is arranged such that rays 212G and 212R are converged by lens 206 and intersect and diverge again before they are incident on lens 204. This limits the lateral separation of the green and NIR rays to the extent that lens 204 can bring them to a common axial focal point.

It is believed that for 532 nm and 1064 nm radiation a spot size difference of about 10% or greater and possibly 15% or greater is achievable in an optical system in accordance with embodiment 34F of the present invention. It is also believed that by grossly undercorrecting lateral color (while continuing to correct axial color) long and short-wavelength treatment-spots can be projected wherein the long-wavelength treatment-spot has a diameter larger than the short-wavelength spot.

There are certain potential limitations of this system. By way of example the axial correction and lateral under/over correction of color may be very difficult to accomplish in a variable magnification system. The maximum spot-size difference will decrease as the difference in wavelengths of the radiation forming the images decreases. Further, if light sources providing radiation in a relatively (relative to lasers) broad spectral bandwidth, such as a filtered discharge lamp are used, edge-definition of the treatment spots will degrade with increasing bandwidth.

It is emphasized here that while optical systems in accordance with the present invention have been described above primarily with respect to providing different treatment-spot sizes for 532 nm and 1064 nm radiation, with the 532 nm treatment-spot size being the greater this should not be construed as limiting the present invention. Those skilled in the art will recognize from the detailed description provided herein that an optical system in accordance with the present invention can be configured to project treatment spots at other wavelengths that can be transmitted along an optical fiber, and to project treatment spots having sizes different from those which are preferred in the Black et al. treatment. An optical system in accordance with the present invention can also be configured to project different-sized, long and short-wavelength treatment spots wherein the long-wavelength spot has the larger size.

In summary, the present invention is described and depicted herein with reference to a preferred and other embodiments. The invention, however, is not limited to those embodiments described and depicted. Rather the invention is limited only by the claims appended hereto.

What is claimed is:

1. An optical system for delivering electromagnetic radiation in first and second different wavelengths to tissue to be treated therewith, comprising:
a first optical subsystem for forming the different-wavelength radiations into beams of first and second different sizes and combining the beams on a common optical path; and
a second optical subsystem arranged to project said combined first and second sized beams onto the tissue to be treated to form overlapping treatment spots thereon of respectively third and fourth different sizes.

2. The optical system of claim 1, wherein said treatment spots are about circular and are concentric.

3. The optical system of claim 1, wherein at least one of said optical subsystems is arranged such that said third and fourth sizes are selectively variable.

4. The optical system of claim 3, wherein the ratio of said third and fourth sizes is about the same.

5. The optical system of claim 3, wherein the ratio of said third and fourth sizes is variable.

6. The optical system of claim 1, wherein the ratio of said first and second sizes is between about 6:5 and 3:1.

7. The optical system of claim 1, wherein said optical subsystem includes first and second optical fibers for delivering respectively said first and second-wavelength radiations, and said first and second optical fibers have respectively first and second exit-face sizes related to said treatment-spot sizes.

8. The optical subsystem of claim 7, wherein said first and second optical fibers also are arranged to transport said first and second radiations from corresponding sources thereof into said first optical subsystem from corresponding sources of said radiations.

9. The optical system of claim 7, wherein said first and second radiations are delivered to said first optical subsystem optical system from corresponding sources thereof along a third optical fiber and said first optical subsystem includes a wavelength selective splitter for extracting said first and second wavelength radiations from said first optical fiber and directing said first and second wavelength radiations into respectively said first and second optical fibers.

10. The optical system of claim 1, wherein said first and second optical paths include optical components arranged for providing first and second different optical powers in said first and second optical paths.

11. The optical system of claim 1, wherein said second optical subsystem includes a first plurality of optical components arranged such that said second optical subsystem has a variable optical power.

12. The optical system of claim 11, wherein said first optical subsystem includes a second plurality of optical components optical arranged such that said first optical path has a variable optical power.

13. Apparatus for delivering electromagnetic radiation in first and second different wavelengths received from corresponding sources thereof to tissue to be treated therewith, comprising:
first and second optical fibers, said first and second fibers having respectively first and second exit-faces of respectively first and second different sizes;
an optical projection system including a plurality of optical components arranged on an optical axis, said optical system arranged with respect to optical fibers such that said first and second exit-faces thereof are located on opposite sides of said optical axis;
said first optical fiber being arranged to direct a beam of said first wavelength radiation from said exit-face thereof into said optical projection system, and said second optical fiber being arranged to direct a beam of said second wavelength radiation from said exit-face thereof into said optical projection system;
components of said optical projection system being arranged such that there is pupil plane between adjacent ones of said optical components and such that the first and second wavelength radiations directed into the system from said optical-fiber exit-faces intersect in said pupil plane; and
wherein, said optical components are further arranged such that an image of said pupil plane is formed on the tissue when the tissue is located at a predetermined working distance from the optical system, said pupil plane image forming a spot of said first wavelength radiation having a third size and a spot of said second wavelength radiation having a fourth size, said spots overlapping each other, and said third and fourth sizes being different and related to said first and second sizes of said fiber-exit faces.

14. An optical system for projecting electromagnetic radiation in first and second different wavelengths received from corresponding sources thereof to tissue to be treated therewith, the first and second wavelength radiations being delivered to the optical system along a common optical fiber and emerging from an exit face thereof, the optical system comprising:
a plurality of optical elements arranged to project an image of the optical fiber exit-face onto the tissue, said optical components being selected and arranged such that lateral color aberration of the optical system is either sufficiently overcorrected or sufficiently undercorrected that the projected image has a first size for said first wavelength radiation and a second size for said second wavelength radiation, said first size being greater than said second size.

15. The optical system of claim 14, wherein said optical components are further selected and arranged such that axial color aberration of the optical system substantially corrected.

16. The optical system of claim 14, wherein said optical components are selected and arranged such that lateral color aberration of the optical system is overcorrected, and said first wavelength is longer than said second wavelength.
